(19) United States
(12) Patent Application Publication
(10) Pub. No.: US 2024/0133806 A1
(43) Pub. Date: Apr. 25, 2024

(54) Device and Method for Optical Coherence Tomography In Laser Material Processing

(71) Applicant: II-VI Delaware, Inc., Wilmington, DE (US)

(72) Inventors: Marcin Kozak, Kleinmachnow (DE); Giovanni Barbarossa, Kleinmachnow (DE)

(21) Appl. No.: 18/073,156

(22) Filed: Dec. 1, 2022

(30) Foreign Application Priority Data
Oct. 20, 2022 (DE) 10 1022 003 907.9

(51) Int. Cl. G01N 21/45; B23K 26/03; G02B 3/00; G02B 27/14

(52) U.S. Cl. CPC G01N 21/453; B23K 26/032; G02B 3/0056; G02B 27/14; G01N 2021/1787

(57) ABSTRACT

A device for monitoring a process in laser material processing, comprising a laser generating a light beam, wherein the light beam may impinge on a lens matrix disposed between the light source and a beam splitter. The lens matrix may comprise microlenses, operable to generate a matrix of light beams from the impinging light beam. Part of the matrix of light beams may be directed to a mirror in a reference arm and part may be directed to an unknown surface in a measuring arm. The reflection of these beams may be used to generate an interference signal to be evaluated.

### CROSS-REFERENCE TO RELATED APPLICATION

This patent application claims priority to and the benefit from German Patent Application DE 10 1022 003 907.9 filed Oct. 21, 2022 at the Deutsches Patent-und Markenamt. The above application is incorporated by reference herein.

### TECHNICAL FIELD

The present disclosure relates to a device and method for optical coherence tomography in laser material processing.

### BACKGROUND

Aspects of the present disclosure relate to a device and method for optical coherence tomography in laser material processing. Various issues may exist with conventional solutions for optical coherence tomography in laser material processing. In this regard, conventional systems and methods for optical coherence tomography may be costly, cumbersome, and/or inefficient.

Limitations and disadvantages of conventional systems and methods will become apparent to one of skill in the art, through comparison of such approaches with some aspects of the present methods and systems set forth in the remainder of this disclosure with reference to the drawings.

### BRIEF SUMMARY OF THE DISCLOSURE

Shown in and/or described in connection with at least one of the figures, and set forth more completely in the claims are a device and method for optical coherence tomography.

These and other advantages, aspects and novel features of the present disclosure, as well as details of illustrated embodiments thereof, will be more fully understood from the following description and drawings.

### BRIEF DESCRIPTION OF THE DRAWINGS

The various features and advantages of the present disclosure may be more readily understood with reference to the following detailed description taken in conjunction with the accompanying drawings, wherein like reference numerals designate like structural elements.

FIG. 1 shows an arrangement for OCT measurements.

FIG. 2 shows an arrangement for measurements using OCT.

### DESCRIPTION

The following discussion provides various examples of semiconductor devices and methods of manufacturing semiconductor devices. Such examples are non-limiting, and the scope of the appended claims should not be limited to the particular examples disclosed. In the following discussion, the terms “example” and “e.g.” are non-limiting.

The figures illustrate the general manner of construction, and descriptions and details of well-known features and techniques may be omitted to avoid unnecessarily obscuring the present disclosure. In addition, elements in the drawing figures are not necessarily drawn to scale. For example, the dimensions of some of the elements in the figures may be exaggerated relative to other elements to help improve understanding of the examples discussed in the present disclosure. The same reference numerals in different figures denote the same elements.

The term “or” means any one or more of the items in the list joined by “or”. As an example, “x or y” means any element of the three-element set {(x), (y), (x, y)}. As another example, “x, y, or z” means any element of the seven-element set {(x), (y), (z), (x, y), (x, z), (y, z), (x, y, z)}.

The terms “comprises,” “comprising,” “includes,” and/or “including,” are “open ended” terms and specify the presence of stated features, but do not preclude the presence or addition of one or more other features.

The terms “first,” “second,” etc. may be used herein to describe various elements, and these elements should not be limited by these terms. These terms are only used to distinguish one element from another. Thus, for example, a first element discussed in this disclosure could be termed a second element without departing from the teachings of the present disclosure.

Unless specified otherwise, the term “coupled” may be used to describe two elements directly contacting each other or describe two elements indirectly connected by one or more other elements. For example, if element A is coupled to element B, then element A can be directly contacting element B or indirectly connected to element B by an intervening element C. Similarly, the terms “over” or “on” may be used to describe two elements directly contacting each other or describe two elements indirectly connected by one or more other elements.

Optical coherence tomography (OCT) is a technology that may be used for high-resolution cross-sectional imaging. OCT uses light and may be used to obtain, for example, cross-sectional images of tissue structure at micrometer scale, in situ and in real time. The use of OCT in combination with catheters and endoscopes may enable high-resolution intraluminal imaging of organ systems.

OCT may act as a type of optical biopsy and may be powerful as an imaging technology for medical diagnostics for use e.g., in ophthalmology.

OCT may also be used for material processing. For example, an OCT configuration may use a single low-coherence light source and detector in combination with a deflection mirror. This technology may generate a single “pixel” that is swept across an entire field, which may be of interest for process monitoring. However, in some instances speed and quality of the measurement data may be limited when sophisticated optics and electronics may be required for data acquisition and processing. In accordance with various embodiments of the invention, monitoring of material processing using OCT may be improved.

For example, micro-optics may be arranged in the optical path of the OCT between the laser source and the beam splitter so that an M×N matrix of independent sub-elements, corresponding to the number of micro-optics, may be used for measurement instead of e.g., a single laser source corresponding to a single pixel.

The sub-elements, in the sense of light beams or pixels, may be combined in a kind of matrix, where each of the sub-elements may be controlled separately. The light may be projected onto the surface to be measured and the reflected light may be collected. The matrix described above may be understood as a grid of many miniature lenses acting individually. These can also be arranged in rows or lines as may be required by a specific application.

The use of such a matrix may allow to obtain M×N measuring points as a snapshot in the sense of capturing an instantaneous state. This may be particularly advantageous for processes that constantly change their state, such as a melting zone in the laser welding process, for example.

Since the size of individual pixels in the matrix may be technologically limited, but individual pixels may be controlled separately or bundled as needed, it may also be possible to create scan programs depending on requirements. One of these scan programs may be a kind of “flash lidar” for melt pools, i.e., a “photo” that may contain substantially all individual pixels instead of a result of many scans over an entire field of view. This may increase the quality of the results and may lead to better quality assurance when welding critical paths, e.g., for parts of a battery for electric vehicles.

Furthermore, in accordance with various embodiments of the invention, a camera may be used to record interference signals of such an M×N matrix.

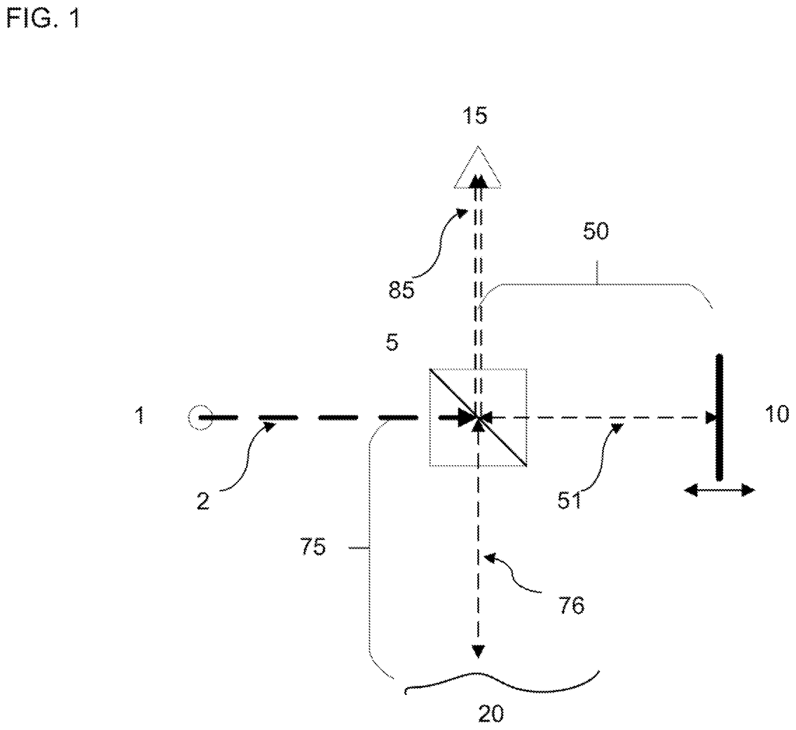

FIG. 1 shows an exemplary arrangement for OCT measurements. A light beam 2 may be generated at a light source 1 with low coherence and may impinge on a beam splitter 5. From the beam splitter 5, an unknown surface 20 of a sample may be illuminated in the measuring arm 75 and light may be reflected by the unknown surface 20 onto the beam splitter 5. The light transmitted through the beam splitter 5 may hit a mirror 10 in the reference arm 50 and may be reflected back by the mirror 10. Reflected sample beam 76 and reference beam 51 may then combine in beam splitter 5 and may interfere when the difference in the paths traveled by the two beams 76, 51 may be less than a coherence length. The interference signal 85 may be recorded by a detector 15 and may then be evaluated. For evaluation, the detector 15 may be coupled to an evaluation unit (not shown). This may be a data processing unit, for example.

Moving the mirror in the reference arm 50 (double arrow) along the beam axis of the light beam 2 emitted from the light source 1, while simultaneously measuring the interference signal 85 may allow axial scanning of the unknown surface 20 of the sample.

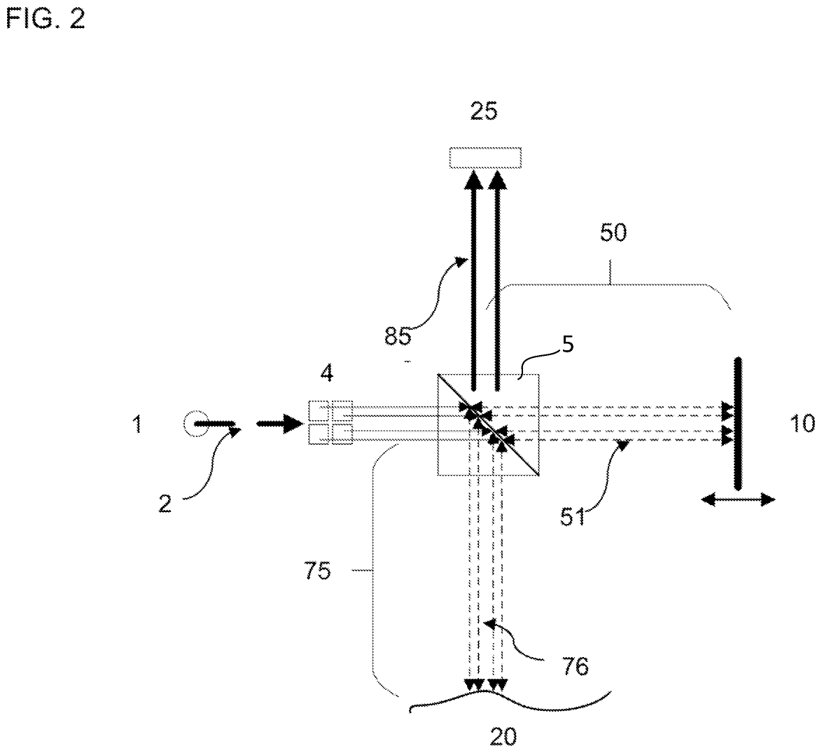

FIG. 2 shows an exemplary arrangement in accordance with various embodiments of the invention. A lens matrix 4 comprising an M×N matrix of microlenses may be arranged between light source 1 and beam splitter 5. A plurality of light beams 2 may thus impinge on the beam splitter 5 and thus a plurality of reference beams 51 may impinge on the mirror 10 in the reference arm 50, as well as a plurality of sample beams 76 may impinge on the unknown surface 20 of the sample in the measuring arm 75.

In accordance with various embodiments of the invention, infrared light may be emitted from the light source, so that, for example, a laser diode may be used as the light source.

A polygonal shape may be used for the microlenses, i.e., a square, rectangular, hexagonal, octagonal, etc. shape may be provided, so that the microlenses may be arranged with substantially no space between them. With respect to the optical properties of the microlenses, the light rays emerging from the microlenses may be substantially parallel so that a matrix having M×N light rays or light spots may thus be obtained.

A camera 25 may used as a sensor instead of a small detector for a single beam. The plurality of beams 51, 76 from reference arm 50 and measuring arm 75, from the M×N matrix may be evaluated accordingly in the interference signal 85, which may also correspond to a plurality of M×N beams from the M×N matrix.

Such an arrangement according to various embodiments of the invention may result not only in a single point that may be viewed and/or evaluated, but in an area with a number of pixels M×N corresponding to the matrix.

In laser material processing, the melt pool may change continuously during an ongoing work process. With an arrangement as described above, it may be possible to continuously monitor a constantly changing surface of the melt pool, or its course front, respectively. For this purpose, so-called LiDAR (Light Detection and Ranging) sensors may be used as a camera, in accordance with various embodiments of the invention. Black and white images may be sufficient for a camera, although a camera for color images may also be used. The frame rate may be preferably above 10 fps (frames per second).

A method and use thereof for monitoring and controlling a process in laser material processing may be implemented, in accordance with various embodiments of the invention. In addition to a pictorial representation of the results of the evaluation of the interference signal, such results may also be used to control or optimize laser material processing. The individual pixels in the matrix may be controlled individually. This means that they may be moved individually in X and Y dimensions, as desirable. For this purpose, for example, the microlenses may then be moved accordingly, which may change the position of the light beam and thus may also change the position of the associated pixel in the interference signal.

Thus, the method according to various embodiments of the invention may be used to control a process of laser material processing and thus may ultimately control it. In this case, it may not just be a matter of quality assurance, but a matter of control.

Other aspects, features and advantages of the present invention will readily be apparent from the following detailed description, which simply sets forth preferred embodiments and implementations. The present invention may also be realized in other and different embodiments, and its various details may be modified in various obvious aspects, without departing from the teachings and scope of the present invention. Accordingly, the drawings and descriptions are to be considered illustrative and not limiting. Additional purposes and advantages of the invention are set forth in part in the following description and will become apparent in part from the description or may be inferred from the embodiment of the invention.

The present disclosure includes reference to certain examples, however, it will be understood by those skilled in the art that various changes may be made and equivalents may be substituted without departing from the scope of the disclosure. In addition, modifications may be made to the disclosed examples without departing from the scope of the present disclosure. Therefore, it is intended that the present disclosure not be limited to the examples disclosed, but that the disclosure will include all examples falling within the scope of the appended claims.

What is claimed is:

1. A device for laser material processing, comprising:
a laser generating a light beam, said light beam impinging on a lens matrix disposed between said light source and a beam splitter;
said lens matrix comprising M×N microlenses, operable to generate a matrix of M×N light beams from said impinging light beam;
said beam splitter directing a first part of said M×N light beams onto a mirror in a reference arm and a second part of said M×N light beams onto an unknown surface in a measuring arm, wherein said first part of said M×N light beams is reflected back from said mirror to said beam splitter and said second part of said M×N light beams is reflected back from said unknown surface to said beam splitter;
said beam splitter operable to generate an interference signal by interfering said first reflected part of said M×N light beams with said second reflected part of said M×N light beams; and
a detector receiving said intereference signal.

2. The device of claim 1, wherein said detector is a camera.

3. The device of claim 1, wherein said detector is a black and white camera or a color camera.

4. The device of claim 1, wherein said microlenses have a polygonal shape such that they are arranged with substantially no space between them.

5. The device according to claim 1, wherein said mirror is coupled to a drive to move said mirror in the direction of a beam path of said light beam.

6. The device of claim 1, further comprising a unit for evaluating said detected interference signals, wherein said detector is connected to the unit for evaluating data.

7. A method for monitoring unknown surfaces in a laser material process, the method comprising the following steps:
generating a light beam with a laser, said light beam impinging on a lens matrix disposed between said light source and a beam splitter;
generating a matrix of M×N light beams from said impinging light beam, using said lens matrix comprising M×N microlenses;
using said beam splitter, directing a first part of said M×N light beams onto a mirror in a reference arm and a second part of said M×N light beams onto an unknown surface in a measuring arm, wherein said first part of said M×N light beams is reflected back from said mirror to said beam splitter and said second part of said M×N light beams is reflected back from said unknown surface to said beam splitter;
generating an interference signal by interfering said first reflected part of said M×N light beams with said second reflected part of said M×N light beams in said beam splitter; and
receiving said intereference signal at a detector.

8. The method of claim 7, wherein said receiving of said interference signal is performed by a camera.

9. The method according claim 7, wherein said received interference signal is evaluated by an evaluation unit connected to said detector.

10. The method according to claim 9, wherein the result of said evaluation is shown as an image on a display.

11. The method of claim 7, wherein said matrix of M×N light beams and thus a matrix of M×N pixels of said interference signal can be individually controlled.

12. The method of claim 11, wherein said individually controlled light beams and said pixels may comprise a movement in the X or Y direction.

13. The method of claim 9, wherein said evaluation is used to control a process in laser material processing.

14. The method of claim 7, said laser material process being a a welding process or a cut material process.

15. The method of claim 11, for monitoring joining processes when joining workpieces by means of a laser beam.
